(19) United States
(12) Patent Application Publication
(10) Pub. No.: US 2026/0120814 A1
(43) Pub. Date: Apr. 30, 2026

(54) SYSTEMS, METHODS, AND DEVICES FOR PREDICTING PHARMACOKINETIC INFLUENCES ON KETOGENESIS FOLLOWING ADMINISTRATION OF TRICAPRILIN

(71) Applicants: CERECIN INC., Denver, CO (US); NATIONAL UNIVERSITY OF SINGAPORE, Singapore (SG)

(72) Inventors: Gisela RAMIREZ, Parkville (AU); Thomas KAASGAARD, Parkville (AU); Benjamin BOYD, Parkville (AU); Matthias Gerhard WACKER, Singapore (SG); Zhuoxuan LI, Singapore (SG); Samuel T. HENDERSON, Golden, CO (US); Murali NAIR C K BALACHANDRAN, Singapore (SG); Bruce H. MORIMOTO, Denver, CO (US); Aikun Julie LIU, Geelong (AU)

(21) Appl. No.: 18/848,355

(22) PCT Filed: Mar. 24, 2023
(86) PCT No.: PCT/US2023/016186

§ 371 Date: Sep. 18, 2024

Related U.S. Application Data
(60) Provisional application No. 63/323,305, filed on Mar. 24, 2022.
(60) Provisional application No. 63/423,349, filed on Nov. 7, 2022.

(51) Int. Cl. G16C 20/30

(52) U.S. Cl. CPC G16C 20/30

(57) ABSTRACT

Systems, methods, and devices predict pharmacokinetic parameters following administration of a formulation, such as a medium-chain triglyceride (MCT). The method includes generating training data indicating human metabolism outcomes of the formulation. The training data includes stomach emptying parameters, a digestion rate, an absorption constant, or a conversion rate with a training target variable being ketone concentration or plasma free fatty acid concentration. The method also includes training a physiologically based biopharmaceutics (PBB) model with the training data and using the PBB model to predict, using the patient data, at least one of a target plasma free fatty acid concentration or a target ketone concentration. Furthermore, as part of a parameter sensitivity analysis of the PBB model, the method varies input parameters of the training data against a simulated mean plasma free fatty acid profile or a simulated mean ketone plasma profile.

### CROSS-REFERENCE TO RELATED APPLICATION

This application claims priority to U.S. Provisional Patent Application No. 63/323,305, filed Mar. 24, 2022, and titled “METHODS FOR PREDICTING PHARMACOKINETIC INFLUENCES ON KETOGENESIS FOLLOWING ADMINISTRATION OF TRICAPRILIN TO HUMANS,” and U.S. Provisional Patent Application No. 63/423,349, filed Nov. 7, 2022 and titled “METHODS FOR PREDICTING PHARMACOKINETIC INFLUENCES ON KETOGENESIS FOLLOWING ADMINISTRATION OF TRICAPRILIN TO HUMANS,” the entireties of each are incorporated herein by reference.

### FIELD

Aspects of the presently disclosed technology relate generally to systems and methods for predicting pharmacokinetics and, more particularly, to predict ketogenesis parameters for Alzheimer's patients.

### BACKGROUND

Alzheimer's disease (AD) is a progressive neurodegenerative disorder and the most common cause of dementia, accounting for approximately 60-80% of all cases. In the United States of America alone, an estimated 6.2 million patients aged 65 and older are affected. Currently, there are several theories regarding the cause and underlying mechanisms of AD. They include the formation of amyloid f3 plaques, neurofibrillary tangles in the neurons as well as chronic inflammation of brain tissues. Many pathophysiological pathways contributing to the disease are yet to be explored.

Investigations have confirmed a reduced expression of glucose transporters in the central nervous system (CNS) of Alzheimer's patients. Also, enhancing their glucose metabolism can contribute to a prolonged lifespan. In this context, the ketones acetoacetate and f3-hydroxybutyrate serve as a primary energy source and provide up to 80% of the brain's energy when glucose is not available. This has led to the development of ketogenic diets to improve the energy supply and achieve functional improvements in patients with AD.

It is with these observations in mind, among others, that various aspects of the present disclosure were conceived and developed.

### SUMMARY

Implementations described and claimed herein can address the foregoing problems by providing a method of predicting pharmacokinetic parameters following administration of a formulation. The method can include generating training data indicating human metabolism outcomes of the formulation; training, by one or more computer devices executing computer-readable instructions, a physiologically based biopharmaceutics (PBB) model with the training data; and/or varying, as part of a parameter sensitivity analysis of the PBB model, input parameters of the training data against a simulated mean plasma free fatty acid profile (e.g., octanoic acid) or a simulated mean ketone plasma profile. Furthermore, the method can include receiving, by the one or more computer devices, patient data corresponding to a patient that receives the formulation perorally; and predicting, with the PBB model using the patient data, at least one of a target plasma free fatty acid concentration or a target ketone concentration.

In some examples, the formulation includes a medium-chain triglyceride (MCT). The MCT can be tricaprilin. The training data can also include a data set based on a gastric emptying model. For instance, the gastric emptying model can include a sine algorithm corresponding to a rhythmic emptying pattern. Additionally, the gastric emptying model can include a first pathway for solid foods and a second pathway for liquid foods; and/or a stomach amplitude parameter, a complete emptying parameter, or a stomach period parameter.

In some instances, predicting at least one of the target plasma free fatty acid concentration or the target ketone concentration includes determining a digestion rate constant obtained by fitting a release profile of the formulation in a presence of lipases while assuming a first-order kinetic process. The training data can include an in vitro parameter being a digestion rate and/or a bioavailability digestion fraction. The training data can also include in vivo parameters being one or more of a metabolized fraction, an absorption constant, a conversion rate, an elimination rate, a volume distribution of free fatty acid, or a volume distribution of ketone bodies. Furthermore, the patient data can include one or more of patient physiological parameters or formulation dosage information.

In some examples, the method can further include modeling a digestion rate using a release profile of the formulation, and can also include obtaining a digestion rate constant based on modeling the digestion rate, wherein predicting at least one of the target plasma free fatty acid concentration or the target ketone concentration can include using, with the PBB model, the digestion rate. Predicting the at least one of the target plasma free fatty acid concentration or the target ketone concentration can include predicting, with the PBB model, a bioavailability of free fatty acid for the patient based on the digestion rate. Furthermore, training the PBB model can include instructing the PBB model to consider an enzymatic conversion of free fatty acid to plasma ketones as non-saturable first-order process.

In some instances, a method of predicting pharmacokinetic parameters following administration of a formulation includes generating training data indicating one or more stomach emptying parameters, a digestion rate, an absorption constant, or a conversion rate with a training target variable being ketone concentration; training, by one or more computer devices executing computer-readable instructions, a PBB model with the training data; receiving, by the one or more computer devices, patient data corresponding to a patient that receives the formulation; and/or predicting, with the PBB model using the patient data, a target ketone concentration.

In some examples, predicting the target ketone concentration includes predicting, with the PBB model and using the patient data, a plasma free fatty acid concentration of the patient. The method can also include performing a validation for the PBB model using clinical trial data which quantifies free fatty acid and ketone bodies from blood plasma of volunteers.

In some instances, a method of predicting pharmacokinetic parameters following administration of a formulation includes generating training data indicating stomach emptying parameters, a digestion rate, an absorption constant, or a conversion rate with a training target variable being plasma free fatty acid concentration; training, by one or more computer devices executing computer-readable instructions, a PBB model with the training data; receiving, by the one or more computer devices, patient data corresponding to a patient that receives the formulation; and/or predicting, with the PBB model using the patient data, a target plasma free fatty acid concentration. In some examples, a treatment action can be taken responsive to predicting the target plasma free fatty acid concentration and/or the target ketone concentration, such as an adjustment to a dosage, concentration, or substance of the formulation.

In some examples, the method further includes predicting a ketone concentration with the PBB model using the patient data, the plasma free fatty acid concentration, and a conversion rate estimate. Training the PBB model can include providing a gastric absorption emptying model having a sine algorithm with an amplitude variable, a period variable, and a complete emptying time variable. Furthermore, the training data can include a first cluster including physiologically based parameters, a second cluster including population parameters, and a third cluster including in vitro parameters.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example biopharmaceutics modeling system including a physiologically based biopharmaceutical (PBB) model.

FIG. 2 illustrates a schematic diagram of an example metabolic configuration for designing a PBB model, which can form at least a portion of the system depicted in FIG. 1.

FIG. 3 illustrates example dispersibility and lipolysis studies of a perorally consumed formulation for the PBB model, which can form at least a part of the system depicted in FIG. 1.

FIG. 4 illustrates example phase I clinical trial data used for model validation,

FIG. 5 illustrates example graphs of a stomach emptying model, which can form at least a part of the system depicted in FIG. 1.

FIG. 6 illustrates a partial parameter sensitivity analysis for a PPB model, which can form at least a part of the system depicted in FIG. 1.

FIG. 7 illustrates graphs of simulated in vivo data for a PBB model including gastric emptying, which can form at least a part of the system depicted in FIG. 1.

FIG. 8 illustrates graphs of simulated in vivo data for a PBB model omitting gastric emptying, which can form at least a part of the system depicted in FIG. 1.

FIG. 9 illustrates graphs of simulated in vivo data for a PBB model showing a digestion rate impact on a PBB model, which can form at least a part of the system depicted in FIG. 1.

FIG. 10 illustrates graphs of simulated in vivo data for a PBB model showing a bioavailability impact on a PBB model, which can form at least a part of the system depicted in FIG. 1.

FIG. 11 illustrates a system including a computing device, which can be the same as or form at least a part of the system depicted in FIG. 1.

FIG. 12 illustrates an example method for predicting pharmacokinetic parameters following administration of a formulation.

### DETAILED DESCRIPTION

Aspects of the present disclosure include systems, methods, and devices for predicting pharmacokinetic parameters (e.g., a plasma free fatty acid concentration, such as octanoic acid concentration, or a ketone concentration) following administration of a formulation comprising a medium-chain triglyceride (MCT), such as tricaprilin. As used herein “medium-chain triglyceride” or “MCT” used herein refers to triglycerides in which all three fatty acid moieties are medium-chain fatty acid moieties. Medium-chain fatty acids (MCFA) are fatty acids that have 6 to 12 carbon atoms. In some aspects, MCT refers to triglyceride with nearly exclusive content of caproic acid (C6:0), octane acid (caprylic acid; C8:0) capric acid (C10:0), decane acid (capric acid; C10:0), or any combination thereof. In some aspects, the MCT is octane acid (caprylic acid; C8:0). In some aspects of the method MCT is tricaprilin. Tricaprilin is a semi-synthetic medium-chain triglyceride (MCT) composed of 8-carbon fatty acids with an aqueous solubility of 0.4 mg/L. Other MCTs can also be used for the dietary management of AD and are within the scope of this disclosure. MCT formulations comprising tricaprilin are used an example throughout this disclosure and it will be appreciated by those of skill in the art that formulations comprising other MCTs can be utilized in the methods disclosed herein. After peroral administration, MCTs, such as tricaprilin, are digested into free fatty acids (e.g., octanoic acid, also known as caprylic acid) by lipases in the gastrointestinal (GI) tract, followed by the absorption of the fatty acid from the duodenum. The free fatty acid, such as octanoate, is then converted to ketone bodies by oxidation in the liver. Medium-chain fatty acids, such as octanoic acid, display preferential absorption and oxidation over long-chain fatty acids. These ketones serve the CNS as an important energy source. Therefore, due to the considerable ketogenic potential of free fatty acids, such as octanoic acid, MCTs such as tricaprilin can provide advantages over previous techniques. Glucose enters the brain through an active transport mechanism that can be saturated. A non-linear relationship between brain and plasma glucose levels can be determined. As compared to the transporter-mediated uptake of glucose into the CNS, there is a linear relationship between the blood and brain concentrations of free fatty acids, such as octanoate. Therefore, increased plasma ketone levels following the administration of MCT (such as tricaprilin) can contribute to a corresponding increase in the brain ketone concentration.

Some pharmacokinetic studies primarily involve direct quantification of the plasma ketone levels after MCT (e.g., tricaprilin) administration. However, the contribution of the sequential individual processes related to digestion of MCTs (for example, tricaprilin) and release of the fatty acid (for example octanoic acid), absorption, and conversion of the free fatty acid into ketone bodies are determined and used by the techniques disclosed herein. Machine-learning modeling such as physiologically based biopharmaceutics (PBB) modeling can leverage the knowledge of the physiological processes involved and, together with selected in vitro and in vivo data sets, generate predictions of the biopharmaceutical behavior of drugs. To cover the complexity underlying the pharmacokinetics of MCT's (such as tricaprilin), a PBB model can be designed based on in vitro digestion studies and human clinical data. The model can be validated using the outcome of a phase I clinical trials. For example, a phase I clinical trial evaluating single-dose administration of the tricaprilin formulation AC-SD-03 to healthy volunteers can be utilized.

Additional advantages of the technology will become apparent from the disclosure herein.

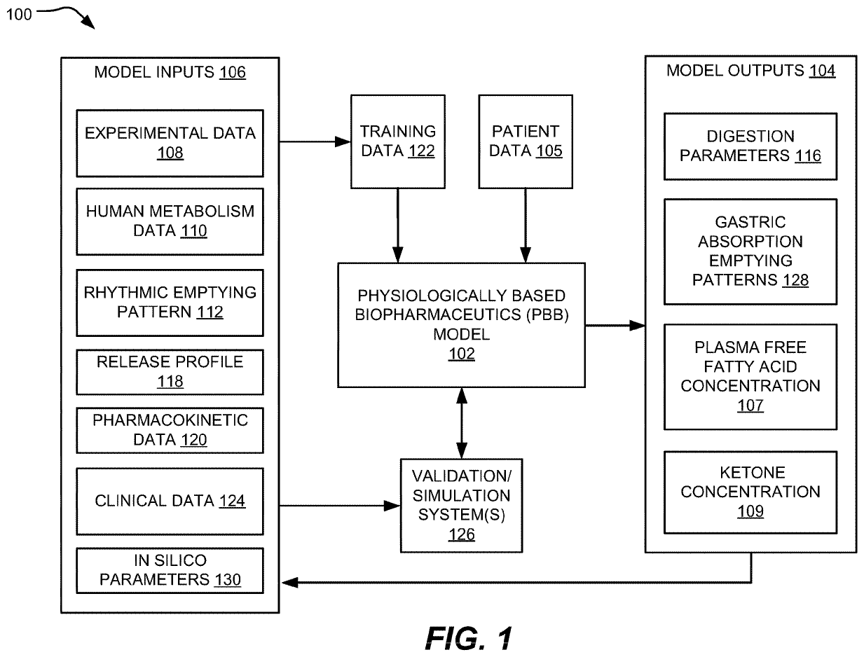

FIG. 1 illustrates an example biopharmaceutics modeling system 100 including a PBB model 102. The PBB model 102 can be developed using MIxEditor 2020R1 (w.g., from Lixoft, Antony, France) to describe an absorption, a metabolism, and/or an elimination of MCT (such as tricaprilin) after preoral consumption. Probability values indicating an impact of each physiological process on ketogenesis, and the generation of the ketone bodies (e.g., f3-hydroxybutyrate and acetoacetate) can be generated as model outputs 104 by the PBB model 102. Once the PBB model 102 is trained and/or validated, the PBB model 102 can receive patient data 105 associated with a target patient. The PBB model 102 can use the techniques discussed herein to analyze the patient data 105 and output, based at least in part on the patient data 105, a predicted plasma free fatty acid concentration 107, such as octanoic acid concentration, 107 and/or a predicted ketone concentration 109. The design and operations of the PBB model 102 are discussed in greater detail below.

The PBB model 102 can receive various model inputs 106 including MCT, such as tricaprilin, experimental data 108 and/or human metabolism data 110. For instance, individual patient profiles can be characterized by a double peak phenomenon. The data can indicate that the MCT formulation, such as the tricaprilin formulation (AC-SD-03), can be administered perorally at a high dose. Consequently, the impact of gastric emptying on the in vivo performance can be assumed based on a gastric emptying model. For instance, gastric emptying can be regulated by multiple factors such as neural circuits and hormones and occurs differently in the fasted and fed state. In the fed state, gastric emptying of solid food may take several hours, as indicated by the data. Another pathway, the stomach road (Magenstrasse), can allow non-caloric liquids to be emptied from the stomach within a much shorter period. Such emptying patterns after the intake of a high-fat homogeneous meal can be observed by the PBB model 102. To interpret this complicated physiological process mathematically, a rhythmic emptying pattern can be incorporated into the PBB model 102. For instance, the rhythmic emptying pattern can include a sine algorithm resembling a regular transport pattern followed by a lag period:

$${k}_{gastric emptying} = amplitude \times sin ( \frac{2 t}{period} ) \qquad ( Equation 1 )$$

The amplitude (a) can represent the maximum transport rate while the duration of the lag phase can be defined by the period (P). After a certain time (h), the gastric compartment can be emptied completely. Suitable simulation parameters can be identified, for instance, using Stella® Architect (v1.5.1, isee systems, Lebanon, USA). A study measuring the gastric content volume after intake of a high-fat homogeneous meal can be used to define one or more gastric emptying parameter ranges. An absolute average fold error (AAFE) can be calculated to compare the model simulations with the clinical data using the following equation:

$$AAFE = {10}^{\frac{1}{n} \times Σ \vert log \frac{{predicted}_{t}}{{observed}_{t}} \vert} \qquad ( Equation 2 )$$

To estimate the digestion of the MCT (e.g., tricaprilin) after entering the small intestine, the in vitro lipid digestion kinetics 114 can be used as model inputs 106. To model a digestion rate 116, the release profile 118 of the fatty acids in presence of lipases can be fitted assuming a first-order kinetic process. A digestion rate constant (k_{d}) can be obtained. Under conditions mimicking GI physiology, the digestion profile can reach a plateau of approximately 83%. The phase I clinical trial can involve quantification of the MCT, such as tricaprilin, as well. A negligible uptake of the undigested triglyceride (Cₘₐₓ=1.05 μ,M) can be observed. It can be determined that the total digestion of the MCT, such as tricaprilin, in the GI tract would be mainly responsible for the overall bioavailability (F) of the free fatty acid (for example, octanoic acid). In total digestion of the MCT (for example, tricaprilin) in the GI tract would be mainly responsible for the overall bioavailability (F) of the free fatty acid (for example, octanoic acid).

Additionally, input parameters are presented in Table 1. Table 1 depicts in silico parameters 130 and parameter ranges which can be used as initial estimates for the in silico analysis of free fatty acid and ketones:

TABLE 1

| Parameter | Range in in silico analysis | Description |
|---|---|---|
| a (h⁻¹) | 0.83-2.48 | Stomach amplitude |
| h (h) | 1.03-3.09 | Complete emptying |
| P (h) | 0.31-0.93 | Stomach period |
| k_{d} (h⁻¹) | 1-5 | Digestion rate |
| F | 83% | Bioavailability |
| | | (digested fraction) |
| p_{metabo} | 5%-9% | Metabolized fraction |
| kₐ (h⁻¹) | 0.23-3.16 | Absorption constant |
| k_{conv} (h⁻¹) | 2.27-2.93 | Conversion rate |
| kₑ (h⁻¹) | 1.38-2.68 | Elimination rate |
| V_{OA} (L) | 40.55-163.26 | Volume of distribution |
| | | of octanoic acid |
| Vₖₑₜₒₙₑ (L) | 12.05-24.41 | Volume of distribution |
| | | of ketone bodies |

To estimate the parameters involved in the distribution, metabolism, and elimination of the free fatty acid (such as octanoic acid), reliable data sources can be identified for generating a training data set to describe plasma pharmacokinetics after intravenous administration of either free fatty acids (such as octanoic acid) or ketones. Pharmacokinetic data 120 related to the MCT (such as tricaprilin) and its metabolites in humans, which can be included in the training data 122 and/or used by a validation system 124 of the biopharmaceutics modeling system, is summarized in Table 2:

TABLE 2

| | Dose | Administration | |
|---|---|---|---|
| Compound | (μmol) | Route | Study Details |
| Tricaprilin | 42500 | Peroral | 20 healthy adults. |
| (AC-SD-03) | | administration | Dose taken 30 min after |
| | | | breakfast. |
| | | | Fasted conditions (≥8 h) |
| | | | (section 2.5) |
| Ketone | 94958 | Intravenous | 12 healthy adults. |
| (Sodium | | injection | Body weight: 52-140 kg. |
| acetoacetate | | | Fasted conditions |
| Ketone | 22842 | Intravenous infusion | 6 healthy males. |
| (DBHB) | 45684 | via arterial, jugular, | Bodyweight in mean ± |
| | 91368 | and femoral venous | SD: 81 ± 9 kg. |
| | | (1 h) | Fasted conditions (10 h) |
| Octanoic acid | 4321.5 | Peroral | 3 ET adults per dose. |
| | 8598.6 | administration | Body weight in mean |
| | 17929 | | (SD): 77.9 (22.4), 77.5 |
| | 28802 | | (13.4), 80.8 (16.4), 64.9 |
| | 71096 | | (1.8), 80.1 (10.2). |
| | | | Fasted conditions |

In Table 2, DBHB is D-f3-hydroxybutyrate; ET is Essential tremor; SD and Standard deviation.

In some examples, ketone plasma concentration can be quantified following an intravenous bolus injection of sodium acetoacetate (n=12). Acetoacetate and D-f3-hydroxybutyrate (n=6), two main components of plasma ketones, can also be quantified upon intravenous infusion of D-f3-hydroxybutyrate at three different doses. Blood free fatty acid (such as octanoic acid) concentration can be measured upon peroral administration of the free fatty acid (e.g, octanoic acid) at five different doses to a patient population (n=3 per dose). This pharmacokinetic data 120 can be analyzed using PKanalix 2020R1 (e.g., Lixoft, Antony, France). These injections, infusions, and/or consumptions of the doses by a target patient can be provided to the PBB model 102 (e.g., as the patient data 105).

In some examples, a one-compartment model may sufficiently describe the pharmacokinetics of free fatty acid (e.g., octanoic acid) and ketones and can later be implemented into a hybrid model. The obtained parameter ranges (minimum, maximum, and mean value over all studies) can be used to define initial estimates in a fixed range when modeling the plasma concentration-time profiles obtained from the phase I clinical trial during model validation. For the development of the pharmacokinetic model, several critical assumptions can be made. They involve the model structure and differential equations used to simulate individual processes. The pharmacokinetic studies provided to the PBB model 102 can involve a wide dose range. Herein, a scalability of the rates between various dose levels and patient populations can be determined or assumed. This can include the enzymatic conversion of free fatty acid (e.g., octanoic acid) to plasma ketones, being considered a non-saturable first-order process.

In some scenarios the PBB model 102 can be written in the Mixtran programming language and executed using Monolix 2020R1 (e.g., Antony, France). The initial parameters and fixed-parameter ranges (e.g., such as those shown in Table 1 and/or the model inputs 106) can be obtained (e.g., from experimentation data and/or literature data).

In the following, population parameters can be obtained for the target population treated with a MCT formulation (e.g., AC-SD-03) using the Stochastic Approximation Expectation-Maximization (SAEM) algorithm. The population parameters can be considered to be log-normally distributed. Additionally, the estimation tasks of the empirical Bayes estimates (EBEs) and the conditional distribution can be performed to calculate the individual predictions and give the uncertainty of the individual parameters and the data for each individual, respectively. The individual clinical data 124 for the free fatty acid (e.g., octanoic acid) and ketones obtained from the pharmacokinetic study can be used for model validation. For instance, the accuracy of the prediction can be assessed by calculating the relative mean absolute prediction error (MAPE, %) using the following equation:

$$MAPE ( \% ) = 100 \times ( \frac{1}{n} \times {E}_{n} \vert \frac{{V}_{O} - {V}_{P}}{{V}_{O}} \vert ) \qquad ( Equation 3 )$$

In equation 3, V_{O} is the observed concentration and V_{P} is the corresponding predicted concentration. The prediction can be interpreted with a MAPE of <10% or 10%-20% as an excellent or good performance. In addition, the model can be evaluated by visual inspection of the goodness-of-fit plots, observations versus individual predictions, individual weighted residuals (IWRES) versus time, and plots of normalized prediction distribution error (NPDE) versus time, as performed by one or more validation/simulation system(s) 126.

In some instances, a partial parameter sensitivity analysis (PPSA) can be carried out by investigating the impact of model input(s) 106 on the outcome of the simulations performed with the PBB model 102. The average PK profiles for both free fatty acid (e.g., octanoic acid) and ketones in humans can be used for simulations. Three different combinations of the input variables can be varied. Specifically, gastric emptying parameters a, h, and P, and in vivo parameters, p_{metabo}, kₐ, k_{conv}, k_{c}, V_{OA}, and Vₖₑₜₒₙₑ can be altered in a range of ±20%. To visualize the influence of the in vitro digestion rate k_{d} on the pharmacokinetic profile, the release rate can be varied to an absolute value of 50%. Further PPSAs for the individual in vivo parameters (p_{metabo}, kₐ, k_{conv}) can be conducted to identify their impacts.

In some examples, simulations with the validation/simulation system(s) 126 can be performed using Simulx 2020R1 (Lixoft, Antony, France) from the PK model with individual estimates produced by Monolix software. One hundred virtual individuals including the plasma concentration-time profiles of free fatty acid (e.g., octanoic acid) and the ketones can be simulated considering single-dose administration of 42,500 μmol of tricaprilin (formulated as AC-SD-03) from 0-16 h. In some scenarios, statistical analysis can be conducted using a t-test on GraphPad Prism 8.

A variety of neurodegenerative disorders result in hypometabolism of the brain due to a significant reduction in the utilization of glucose. Ketogenesis can offer an alternative energy source and can have beneficial effects on the cognitive functions of Alzheimer's patients. The medium-chain triglyceride, for example tricaprilin, is metabolized to ketone bodies, capable of providing 80% of the energy required by the CNS. Other systems have struggled to determine influences on the bioavailability of these ketone bodies. To this end, the PBB model 102 can be a suitable in silico model developed to quantitatively describe various influences on ketogenesis and the effect of physiological parameters on pharmacokinetics. In vitro digestion studies of MCTs (such as tricaprilin) in gastric and intestinal medium can provide a glimpse at the expected degradation rates in the human intestine. Other in vivo parameters with potential impact on the ketone body concentration can be estimated based on information obtained from the literature. A phase I clinical trial evaluating the MCT formulation (such as AC-SD-03) in healthy volunteers can provide the pharmacokinetic data required for model validation.

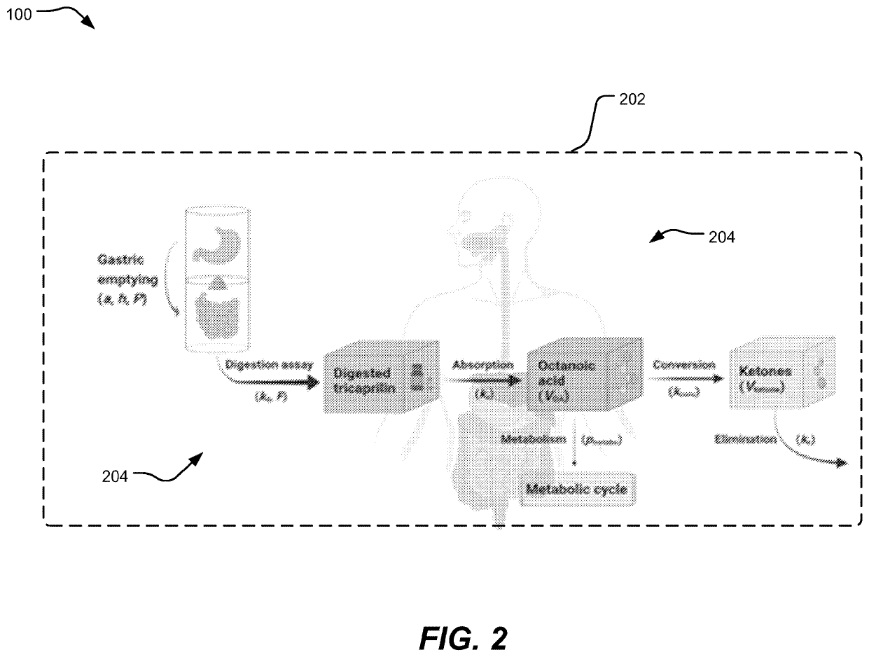

Turning to FIG. 2, a schematic diagram 202 of a metabolic design configuration 204 for designing the PBB model 102 is depicted.

As shown in FIG. 2, the PBB model 102 can be used for in silico analysis and data simulation. The formulation can enter the stomach where the MCT (such as tricaprilin) is released and then transits into the duodenum. The MCT (such as tricaprilin) can be digested into free fatty acids (such as octanoic acid from tricaprilin), followed by the absorption of the fatty acids in the intestine and subsequent metabolism/conversion. A fraction of the absorbed free fatty acid (e.g., octanoic acid) can be converted to ketone bodies followed by elimination from the body. A certain fraction can enter the metabolic cycle and disappears from the blood plasma.

As previously indicated, the formulation can undergo gastric transit before the MCT (e.g, tricaprilin) reaches the intestine. Dissolution and lipolysis of the MCT formulation (for example, AC-SD-03) can lead to the release of free fatty acid (e.g., octanoic acid) that is then absorbed into blood circulation. Afterward, the conversion of free fatty acid (e.g., octanoate) to ketone bodies takes place. This can involve a certain loss to other metabolic pathways including the Krebs cycle. The ketone bodies can appear in the blood plasma where they serve as an energy source for the brain and other tissues. The clinical trial used for model validation can involve the quantification of free fatty acid (such as octanoic acid) and ketone bodies from the blood plasma of healthy volunteers. In the following, the in vitro and in vivo data sources used for model design and validation will be discussed in more detail.

In some examples, an initial dispersibility study can be performed to evaluate the physical stability of a MCT formulation (such as AC-SD-03) in the gastric medium (pH 1.2, 37° C.). It can provide more information on the physical state of the dispersion in the stomach which might affect gastric emptying. Considering that multiple peaks can be observed during the first 4 hours post-administration, this period can be investigated. Other time periods could also be investigated based on the observed peak for the tested MCT formulation.

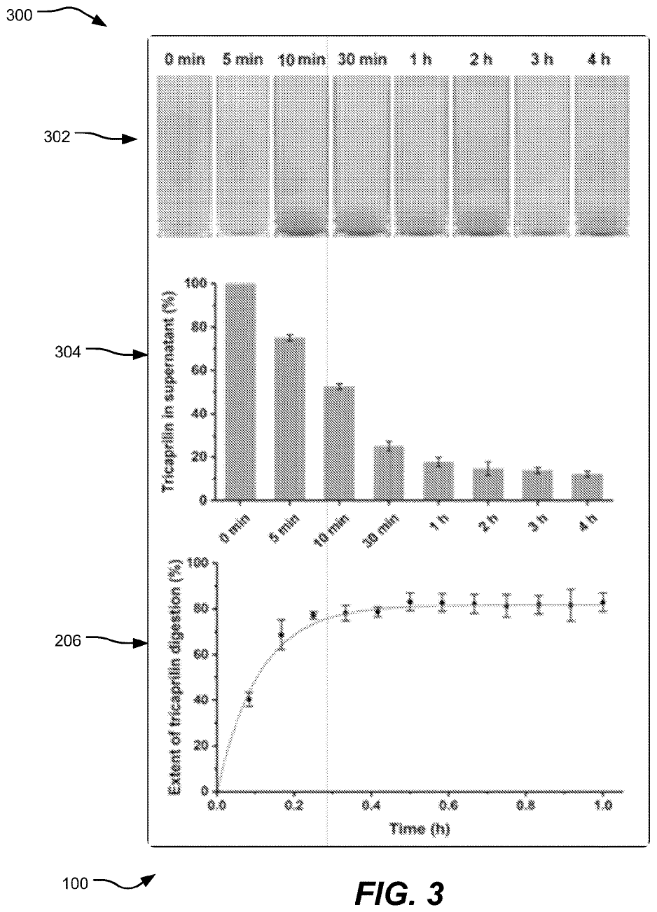

FIG. 3 shows dispersibility and lipolysis studies of AC-SD-03 formulation in 50 mM phosphate buffer (pH 1.2) at 37° C. Images 302 are photographs showing sedimentation of AC-SD-03 formulation within 4 h. Any of the information from these studies can be used as the model inputs 106 and/or for validating the PBB model 102

As depicted in FIG. 3, graph 304 shows percentages of tricaprilin amount in the supernatant of AC-SD-03 suspension within 4 h (n=3). Graph 306 depicts a digestion profile of AC-SD-03 expressed as the percentage of digestion per time. The solid line represents a first-order kinetic fit. Data are presented as mean±standard deviation (SD) (n=3).

In some examples, sedimentation of the MCT formulation (such as AC-SD-03) can occur quickly, as shown in FIG. 3 at images 302 and graph 304. Although no creaming or phase separation of the oil from the water phase can be observed, a significant amount can be sedimented with the insoluble excipients. The experiment can be carried out without agitation. Hence, sedimentation may be overestimated. Still, the adsorption of the MCTs (e.g., tricaprilin) to the solid silica particles may lead to considerable gastric retention. The first dose can be given with a total volume of 300 mL resulting in low viscosity. Accordingly, the major fraction of MCTs (e.g., tricaprilin) likely traveled the stomach road from the fundus through the center of the antrum to the duodenum. After 1 h repeated water intake probably resulted in an additional “washout” of the drug. In this example, a more stable dispersion system can therefore increase the amount of tricaprilin delivered through this pathway.

As a next step, the digestion of the MCT formulation (such as AC-SD-03) in vitro can be measured. The formulation can be exposed to a gastric medium followed by lipolysis in a medium simulating intestinal conditions. The latter can result in the liberation of fatty acids expressed as the percentage of digested free fatty acid (e.g., tricaprilin), as shown at the graph 306. For each mole of tricaprilin, three moles of titratable fatty acids can be released. Within the first 5 min, 40±3% of tricaprilin can be converted to octanoic acid, followed by a gradual reduction in the digestion rate. After 15 min, the digestion profile plateaued at 83±4%. Assuming rapid absorption from the intestine, the digestion rate (kd) and extent (F) of lipolysis are mainly responsible for the bioavailability of octanoic acid. In simulations performed by the validation/simulation system(s) 126, this fraction (83%) can be used as a baseline value when simulating different bioavailabilities. On a side note, there can be a certain probability that undigested tricaprilin can be absorbed into blood circulation as well. However, one estimated rate can be 30-52 gmol/h for this process. This corresponds to less than 0.5% of the total dose during the first 4 hours. Also, negligible amounts of tricaprilin can be found in blood circulation during the phase I clinical trial indicating a minor relevance of this pathway as well. Metabolic processes such as the hepatic conversion of tricaprilin would generally result in the formation of octanoic acid, subsequently converted to ketone bodies.

As outlined previously, the digestion parameters k_{d} and F, which have a strong influence on the absorption of free fatty acid (e.g., octanoic acid), can be determined and used by a digestion parameter model of the PBB model 102. Fitting the in vitro lipolysis profile with a first-order kinetic model (as shown in graph 306 of FIG. 3) resulted in a digestion rate of 4.1 h-1. However, some evidence suggests that the in vitro system does not resemble the physiological process and even higher digestion rates can be expected in vivo. Also, pre-hydrolysis of medium-chain triglycerides by gastric lipase may increase digestion. With respect to potential differences between the in vitro and in vivo lipolysis, digestion parameters in a range from 1-5 h-1 can be determined. Accordingly, the PBB model 102 can fit individual profiles with approximately 20% higher digestion rates. Also, according to the observations of the in vitro experiment, lipolysis of 83±4% of the initial dose can be estimated. This comes with the uncertainties of the in vitro experiment. Therefore, higher and lower bioavailabilities in the virtual clinical trial can be defined, as discussed below.

In some examples, a gastric emptying model can be generated and used by the PBB model 102. By way of a non-limiting example, MCT formulation AC-SD-03 is a powder formulation with high tricaprilin content. The elevated dose together with the low density of the drug promoted the influence of gastric emptying on the pharmacokinetics. A heterogeneous distribution of tricaprilin partially bound to the gastric content can be more than likely. Accordingly, an algorithm can be integrated to simulate these emptying patterns 128. Training data 122 indicating measurements in the change in gastric content volume after the intake of high-fat homogeneous meals in humans can be used. This can be assumed to resemble the intake of the standard breakfast together with tricaprilin by the PBB model 102. A sine algorithm can be used to simulate both, a slow emptying pattern without additional water intake, as well as a repeated “washout” of tricaprilin. The latter would be a consequence of the clinical protocol allowing water intake ad libitum 1 h post-administration. Suitable parameter ranges can be identified by fitting the literature data. An AAFE of 1.11 can indicate an optimal representation of the profile. The parameters used by the algorithm can include the amplitude (a) and period (P) of the sine curve as well as a time of complete emptying (h). They can be 1.65 h\hat{ }1, 2.03 h, and 0.62 h, respectively. Parameter variations within a range of ±50% of the initial value can be estimated (Table 1).

In some examples, the PBB model 102 can generate and/or use a free fatty acid (such as octanoic acid) absorption and metabolism model. The training data 122 can include measurements of the pharmacokinetics of the free fatty acid (such as octanoic acid) after peroral administration at various dose levels with absorption rate constants ranging from 0.23-3.16 h-1. Poor absorption would likely reduce the impacts of gastric emptying on pharmacokinetics and bioavailability. However, for free fatty acids (such as octanoic acid), the absorption rates can be sufficiently high to guarantee complete absorption within the intestinal transit time. Still, the absorption rate can be seen as one of the parameters of influence and further evaluated in the PPSA.

In some examples, the PBB model 102 can include a ketone bodies model for determining ketogenesis from free fatty acids (such as octanoic acid) and pharmacokinetics of ketone bodies. The conversion rate constants of free fatty acids (such as octanoic acid) to ketone bodies (k_{conv}) can be measured in rats and/or humans. To estimate this parameter, human clinical trials can be used as the training data 122. The plasma ketone levels observed after infusion of sodium caprylate in male Sprague-Dawley rats can be considered as a confirmation of first-order kinetics without using this rate for further analysis (e.g., as used by the validation/simulation system 126). The conversion rate of the MCT (for example, tricaprilin) k_{conv} can be estimated based on the elimination rate of the free fatty acid (such as octanoic acid in the case of tricaprilin). Here, ketogenesis can be considered to be the main metabolic pathway. A certain loss to other biochemical pathways can be determined. The Krebs cycle harnesses the available chemical energy of acetyl coenzyme A, an intermediate of octanoic acid, into nicotinamide adenine dinucleotide (NADH) serving as an energy source. Also, f3-hydroxy f3-methylglutaryl-coenzyme A can be used in the synthesis of cholesterol. High sensitivity of the model to either the conversion rate k_{conv} or the metabolized fraction p_{metabo} might reduce its predictive power. Therefore, the PBB model 102 systematically analyzed their sensitivity in a partial parameter sensitivity analysis (PPSA).

In some scenarios, the PBB model 102 can perform a simulation of human pharmacokinetics, for instance, using the validation/simulation system 126. The PBB model 102 can bridge the knowledge gaps between the administration of a MCT (such as tricaprilin) and the appearance of ketone bodies in the blood plasma. Breaking this process down into individual steps followed by partial validations can be one of the objectives of the PBB model 102. The final structure can include simulations of gastric emptying in the fed state with and without additional water intake. A dispersibility study can confirm the formation of a stable low-viscosity emulsion and, during the in vitro lipolysis experiments, for example, more than 80% of tricaprilin can be rapidly digested into octanoic acid. The absorption and conversion rate constants as well as the volume of distribution of the free fatty acid (such as octanoic acid) (VOA) can be obtained by the PBB model 102 by performing the pharmacokinetic analysis of the clinical data.

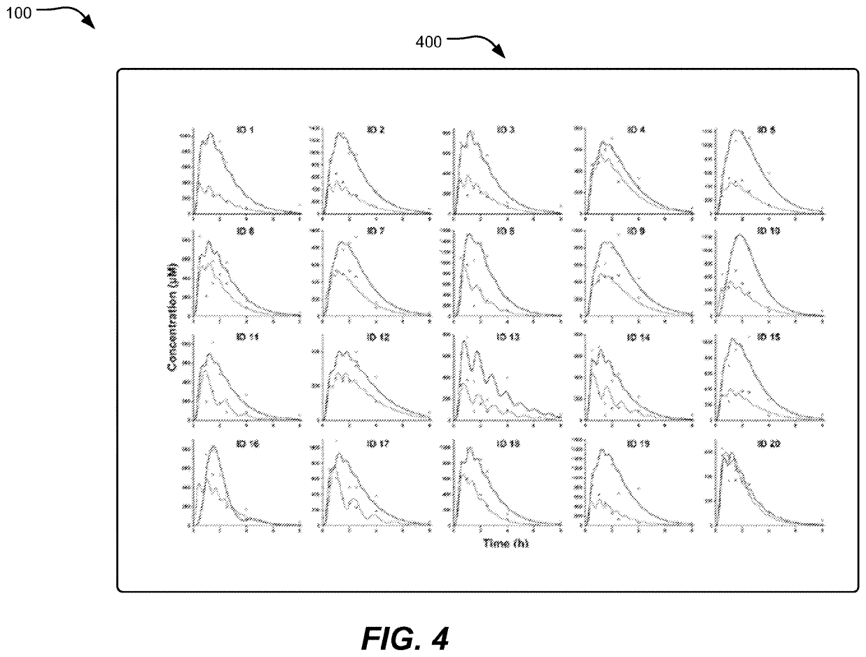

In some examples, as depicted in FIG. 4, the training data 122 can include phase I clinical trial data 400 that provides plasma concentrations of the free fatty acid (in this example, octanoic acid) and ketone bodies in 20 healthy volunteers over time. For model validation, the pharmacokinetic profiles of up to 20 subjects can be used. In silico individual simulations of octanoic acid are shown as violet lines and ketones are shown as black lines in comparison to in vivo pharmacokinetic data of octanoic acid (blue dots) and ketones (red dots). While the model structure still provides a variety of possible situations for this simulation, the fixed parameter ranges can improve estimations by weighing each influence. Expectedly, certain subjects still led to a high prediction error. However, the overall performance of the PBB model 102 for the phase I clinical trial can be acceptable. The individual parameters can be narrowly distributed with the strongest variation in the volume of distribution of the free fatty acid (e.g., octanoic acid). The prediction errors are summarized in Table 3. Table 3 shows the mean absolute prediction errors (MAPE) s of the PBB model 102 for simulations of the human clinical data for all subjects.

TABLE 3

MAPE

| ID | Octanoic acid | ketones |
|---|---|---|
| 1 | 19.92 | 4.78 |
| 2 | 8.69 | 13.86 |
| 3 | 3.73 | 5.28 |
| 4 | 17.13 | 19.02 |
| 5 | 11.84 | 10.52 |
| 6* | 37.13 | 15.27 |
| 7* | 26.35 | 21.62 |
| 8* | 25.81 | 15.83 |
| 9 | 16.14 | 17.41 |
| 10 | 7.06 | 2.91 |
| 11* | 49.02 | 17.26 |
| 12 | 3.62 | 9.36 |
| 13 | 11.71 | 1.00 |
| 14* | 38.68 | 9.09 |
| 15 | 3.92 | 14.35 |
| 16 | 8.35 | 19.38 |
| 17 | 2.48 | 19.27 |
| 18 | 12.09 | 9.77 |
| 19 | 9.36 | 18.65 |
| 20 | 5.66 | 4.71 |

The absence of systematic bias can be confirmed by visualizing individual weighted residual (IWRES) and normalized prediction errors (NPDE) versus time post-dose. The mean can be not significantly different from zero (p=0.06 for IWRES and 0.09 for NPDE, respectively). Each analysis can indicate an accurate prediction.

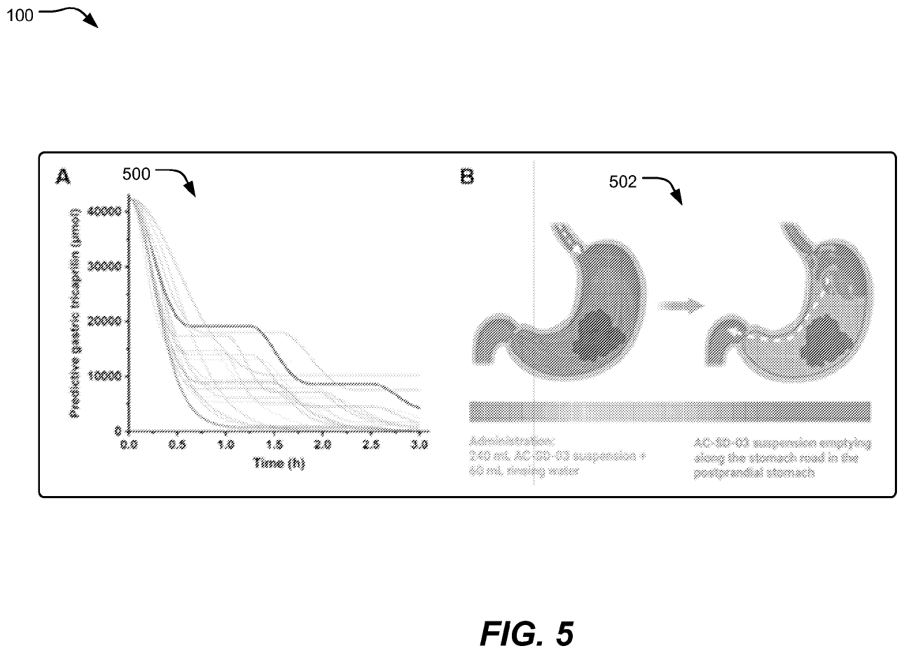

In some examples, gastric emptying represents a strong influence on pharmacokinetics and can be difficult to be simulated. For example, initially, the administration of AC-SD-03 in 300 mL of water potentially can lead to rapid emptying along the stomach road. The emptying patterns corresponding to the 20 healthy volunteers are presented in FIG. 5

FIG. 5 depicts validation techniques of the PBB model 102. Graph 500 depicts predictive tricaprilin amount in the stomach of 20 subjects within 3 h after administration of AC-SD-03. The bold curves (green, blue, and red) represent the typical cases where the subjects had different gastric emptying patterns with different water intakes during the trial. Schematic 502 depicts the emptying of AC-SD-03 suspension from the fed stomach along the stomach road (created with biorender.com).

In some examples, a first emptying can affect 50-100% of the total dose. With water intake not being permitted only 1 h after administration of tricaprilin, for some patients and at later time points, additional water intake can be assumed. Therefore, a model algorithm with the flexibility of simulating various gastric emptying patterns can be used. Emptying of caloric content can occur much slower leading to a plateau (e.g., graph 500 of FIG. 5). The causes for the emptying patterns observed 1 h after the intake of tricaprilin may be due to either the heterogeneous distribution of tricaprilin in the stomach or further liquid intake.

In some scenarios, considering that all population parameters can be within the initial ranges and prediction errors can remain at a low level, initial values (Table 1) can provide an estimate of the physiological and formulation-related influences. This leads to the following conclusions. Once tricaprilin reaches the small intestine, with a rate of 1.7 h\hat{ }1, the absorption of fatty acids can become the rate-limiting step. Also, there can be more significant fluctuations in the absorption rate between different individuals as compared to the much faster lipolysis (4.6 h-1) and ketogenesis (2.6 h\hat{ }1). The conversion of octanoic acid in the liver can play a minor role in the availability of the active form of tricaprilin. One of the main influences can be related to the drug formulation which, in most patients, results in certain retention of tricaprilin in the stomach. These observations can be systematically evaluated in the PPSA, and/or can be provided to the PBB model 102 as model inputs 106 or as part of a validation process.

In some examples, a partial parameter sensitivity analysis (PPSA) can be performed. Model parameters can come with considerable errors. However, simulations based on in vitro data are expected to have the highest uncertainty. A PPSA elucidates parameters with a strong influence on simulations. An alignment between high uncertainty and model sensitivity in one or more parameters can reduce the predictive power.

For instance, the input parameters can be initially divided into three clusters. To identify combinations with major impact, alterations in a range of 20% can be made for physiologically based parameters (e.g., gastric emptying) and population parameters (e.g., elimination rate). The in vitro parameters (e.g., digestion rate) can be altered in a wider range.

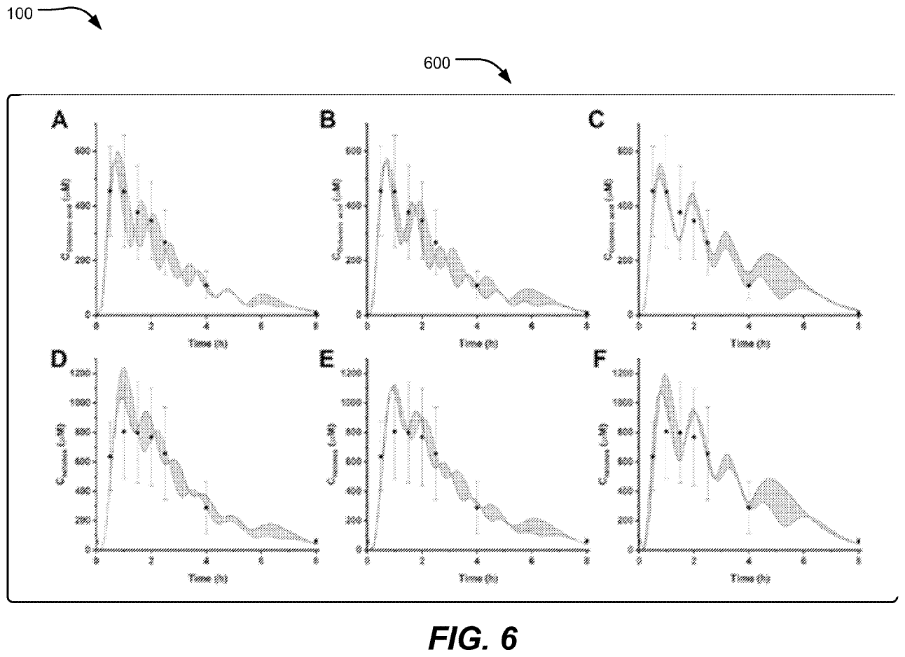

FIG. 6 depicts a partial parameter sensitivity analysis 600 of the in silico model applying incremental changes to (A and D) gastric emptying parameters (a, h, P), (B and E) in vitro parameters kd and F, and (C and F) in vivo parameters (p_{metabo}, kₐ, k_{conv}, kₑ, VOA and Vₖₑₜₒₙₑ). These parameters can be varied against the simulated mean (A, B and C) octanoic acid and (I), E and F) ketone plasma profiles. Observed plasma concentrations can be plotted as symbols with error bars representing standard deviations (n=20).

In some examples, the influences of systematic parameter changes on the simulated pharmacokinetic profiles of octanoic acid and ketones are presented in FIG. 6. The corresponding pharmacokinetic parameters (Cₘₐx, Tₘₐₓ, AU_{Call}) can be included, and the model can be sensitive to the gastric emptying parameters (a, h, and P), the in vitro parameters (k_{d} and F) as well as the in vivo parameters (p_{metabo}, kₐ, k_{conv}, kₑ, V_{OA} and Vₖₑₜₒₙₑ) when they are altered together.

In some instances, the gastric emptying can have the strongest impact on Cₘₐₓ while the in vitro as well as in vivo parameters specifically influenced AU_{Call}. Changes in the individual parameters can further resolve a strong influence of the absorption rate (kₐ). Without alteration, the high metabolic capacity of the liver (represented by k_{conv}) did not become rate-limiting to ketogenesis and can be overpowered by formulation-related parameters. Changing either the enzymic conversion rate (k_{conv}) or the metabolic ratio (p_{metabo}) can have little or no effect on pharmacokinetics. Still, they may be more important for some patient populations, e.g., due to reduced liver function. With the absorption rate being lower than the estimated digestion and conversion rates, it can act as a gatekeeper to the ketone concentration.

In some examples, as a next step, virtual clinical trials can be carried out simulating the expected pharmacokinetics in a larger population. Here, the influences of digestion rate (k_{d}), the extent of intestinal digestion (F), and gastric emptying individually can be determined.

In some instances, in silico predictions of pharmacokinetics can be made by the PBB model 102. For instance, the phase I clinical trial can be used for model validation, which includes a small number of subjects (n=20) only. Accordingly, additional simulations of the expected performance of AC-SD-03 in a larger population can be carried out (n=100).

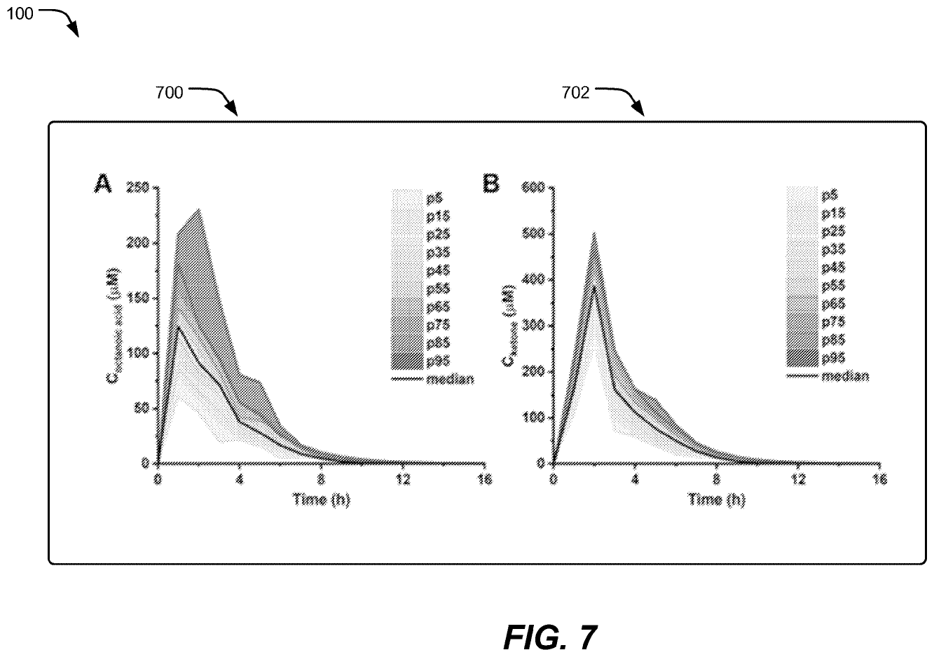

FIG. 7 depicts a first graph 700 of simulated in vivo data for octanoic acid; and a second graph 702 of simulated in vivo data for ketones. The blue bands show percentiles of the simulated data for all individuals and all replicates computed. The solid black curves refer to the median.

As depicted in FIG. 7, the biopharmaceutics modeling system 100 can provide a prediction of the plasma concentration-time profiles of octanoic acid and ketones expected in 100 virtual subjects. The medians of Cₘₐₓ, Tₘₐx, and AU_{Call} obtained from these simulations can be 124.7 μM, 1 h, and 388.2 μM·h for octanoic acid and 387.2 μM, 2 h, and 999.2 μM·h for ketones, respectively. While the powder formulation can lead to higher variability in the plasma octanoic acid concentrations, the plasma ketone levels can remain within a narrower range indicating slight influences of the conversion rate.

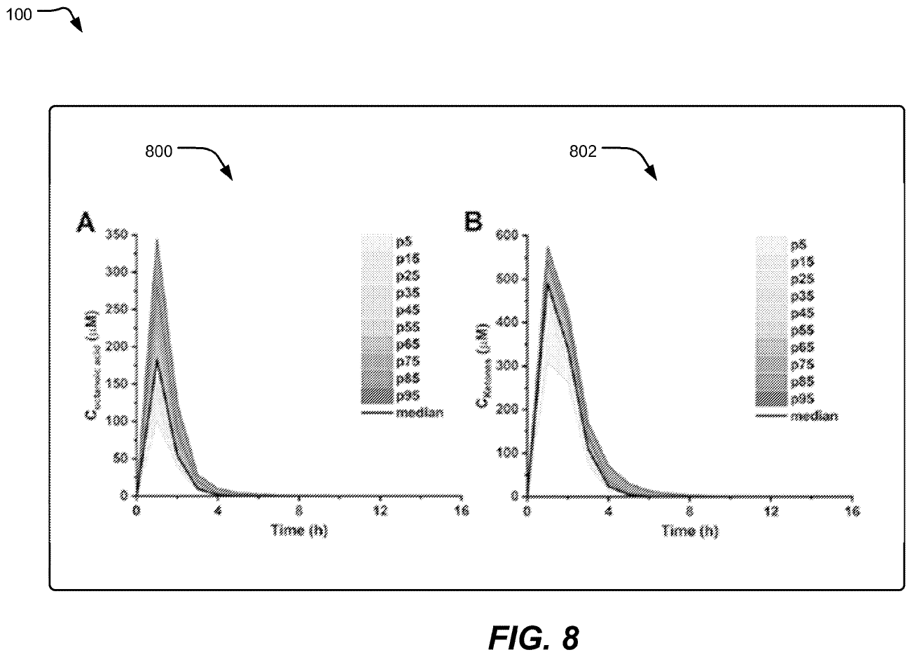

FIG. 8 depicts a first graph 800 of simulated in vivo octanoic acid; and a second graph 802 of simulated ketone concentrations using a physiologically-based biopharmaceutics model without gastric emptying. The blue bands show percentiles of the simulated data for all individuals and all replicates computed. The solid black curves refer to the median.

In some examples, several formulation parameters can be expected to have a considerable effect on the plasma ketone levels. These can include gastric emptying as well as the rate and extent of lipid digestion. The impact of gastric emptying on the in vivo fate of AC-SD-03 can be investigated by removing/omitting the gastric compartment from the PBB model 102 completely. The outcome is presented in FIG. 8. The simulations can indicate that gastric emptying reduces the plasma concentrations considerably and particularly the Cₘₐₓ (70% and 26% increases for octanoic acid and ketones, respectively). A comparison of predictions using the PBB model 102 with and without gastric emptying is presented in FIG. 8. Expectedly, the Tₘₐₓ decreased from 2 to 1 h while AU_{Call} of octanoic acid and the ketones remained almost identical. The Cₘₐₓ can be a strong indicator for a sufficient supply of the brain with ketone bodies. Hence, maximizing the peak concentration would most likely contribute to favorable effects in the treatment of AD.

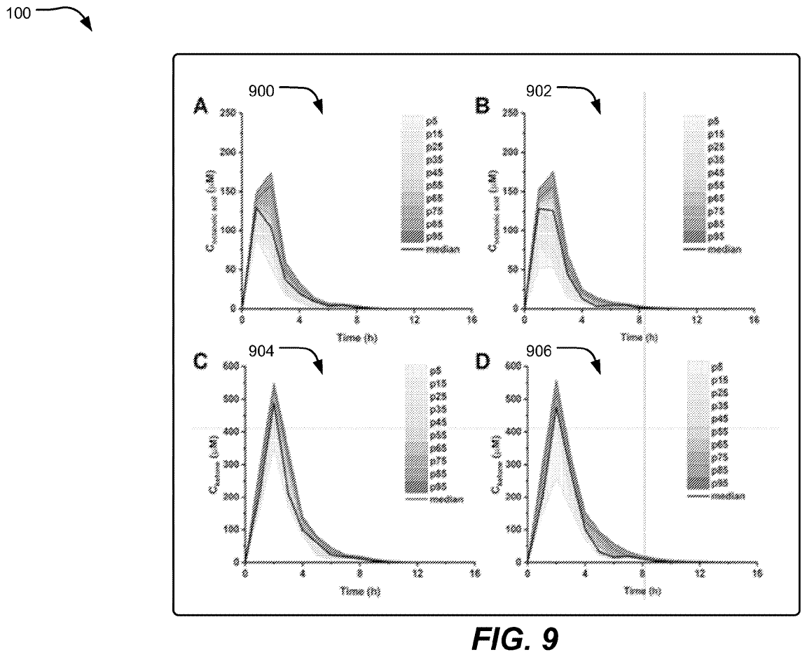

FIG. 9 depicts a first graph 900 and a second graph 902 showing an impact of digestion rate (k_{d}) on in vivo octanoic acid; and a third graph 904 and a fourth graph 906 showing their impact on ketone concentrations. An increase of 50% for k_{d} is depicted in the first graph 900 and the third graph 904; and a 100% for k_{d} is depicted in the second graph 902 and the fourth graph 906. The blue bands show percentiles of the simulated data for all individuals and all replicates computed. The solid black curves refer to the median.

In some examples, further simulations can determine the impact of the digestion rate (k_{d}) on the plasma ketone concentrations. The digestion rate can be increased by 50% and 100%. When increasing k_{d} by 50%, as shown in FIG. 9, a significant increase in the plasma ketone levels can be observed. The median Cₘₐₓ and AU_{Call} can be calculated as 129.5 μM and 311.5 μM·h (octanoic acid) and 488.7 μM, and 1126.5 μM·h (ketones), respectively.

In some scenarios, considering the outcome of the dispersibility study, an increase in the digestion rate may be, for instance, achieved by developing a more stable liquid emulsion system with optimized dispersibility and physical stability. A higher surface area can provide a larger fraction of tricaprilin to be digested at a higher rate.

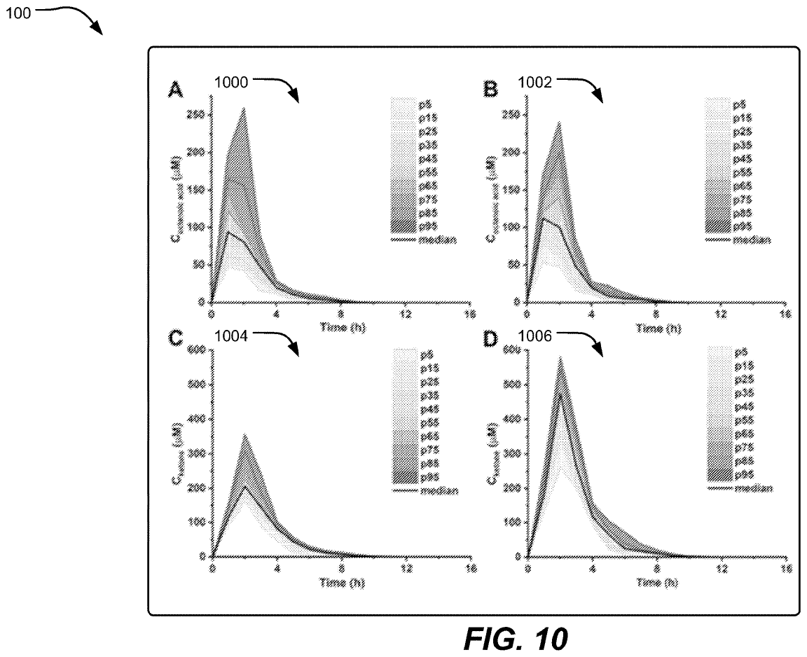

FIG. 10 depicts a first graph 1000 and a second graph 1002 showing an impact of bioavailability, (e.g., tricaprilin digestion (F) on in vivo octanoic acid and, in a third graph 1004 and a fourth graph 1006, on ketone concentrations. The first graph 1000 and the third graph 1004 are with F set at 50%; and the second graph 1002 and the third graph 1004 are with F set at 100%. The blue bands show percentiles of the simulated data for all individuals and all replicates computed. The solid black curves refer to the median.

In some examples, the extent of lipolysis on bioavailability can be evaluated in silico. The degradation of the MCT (e.g., tricaprilin) can be mainly responsible for the fraction of the initial dose becoming available in the blood plasma. With respect to AC-SD-03, a simulation can be performed of an increase in the bioavailability using the digested fraction found in vitro (83%) as a base value and a maximum increase of 17% in bioavailability can be observed (corresponding to 100% bioavailability). Further assuming that an estimation of the absolute bioavailability can be inaccurate, even higher values can be possible, as shown in FIG. 10. However, considering all aspects, limited dispersibility and incomplete lipolysis of AC-SD-03 can remain the most critical formulation parameters limiting bioavailability.

Accordingly, additional approaches can focus on formulation strategies that enhance the rate and extent of digestion and reduce the impact of gastric emptying.

In some examples, an investigation/analysis with the biopharmaceutics modeling system 100 describes and explains ketogenesis after the intake of tricaprilin using a PBB model 102. The simulations can accurately predict the in vivo fate of the spray-dried powder formulation in humans. Estimating physiological and formulation-related influences on pharmacokinetics, the extent of tricaprilin digestion can have a strong impact on bioavailability. Not only can an incomplete in vitro lipolysis be observed, but also the dispersibility study can provide further evidence that a certain fraction of tricaprilin is adsorbed to the insoluble excipients of AC-SD-03. Additionally, the heterogeneous distribution and adsorption of tricaprilin to the gastric content may be mainly responsible for the strong influence of gastric emptying on pharmacokinetics. A considerable delay in the absorption together with a lowered peak plasma concentration of the ketone bodies can be observed. Once octanoic acid reaches the small intestine, the absorption of fatty acids rather than the enzymatic conversion can have a certain gatekeeper effect on Cₘₐₓ of ketones. Even at high doses, the enzymatic capacity can avoid limiting the plasma concentration. When formulating the medium-chain triglyceride tricaprilin, formulation strategies can be aimed at the reduction of the gastric transit time as well as an improved dispersibility to enhance the digestion process. The spray-dried powder formulation can be at least partially processed as a low-density solid promoting the effects of gastric emptying and reducing the extent of its digestion.

In some examples, the biopharmaceutics modeling system 100 can use various materials to perform the techniques disclosed herein. For instance, the spray-dried tricaprilin powder formulation (AC-SD-03) can be composed of 40% of the active pharmaceutical ingredient tricaprilin and 60% of inactive ingredients including Kolliphor RH40 (BASF, Germany), glyceryl monooleate (Abitec Corp., USA), soybean phosphatidylcholine (Lipoid LLC, USA), polyvinyl pyrrolidone (JRS Pharma Gmbh & Co. KG, Germany), Cabosil® fumed silica (Koninklijke DSM N.V., The Netherlands), sucralose, potassium acesulfame, and flavoring agents. It can be manufactured by Lonza (Oregon, USA) with permission from Cerecin Inc. (Singapore). Octanoic acid (>99% purity) can be purchased from Nu-Chek Prep Inc. (Minnesota, USA). Sodium dihydrogen orthophosphate anhydrous (98% purity) can be purchased from Chem Supply (SA, Australia). Methanol (LiChrosolv) can be purchased from Merck Millipore (Bayswater, Australia). Sodium hydroxide pellets (>97% purity) can be purchased from Ajax Finechem (NSW, Australia). Trifluoroacetic acid (HPLC, >99%) and 4-bromophenylboronic acid (4-BPBA, >95% purity) can be purchased from Sigma-Aldrich (St. Louis, USA). FaSSIF (fasted state simulated intestinal fluid) can be purchased from Biorelevant.com Ltd (London, United Kingdom). USP-grade pancreatin extract from porcine pancreas can be purchased from Southern Biologicals (VIC, Australia). The triglyceride quantification colorimetric kit can be purchased from Sigma-Aldrich Pte. Ltd. (MO, United States). Distilled and deionized water (18.2 Mil/cm at 25° C.) can be obtained from a Millipore water purification system (Billerica, USA). It will be appreciated by those of ordinary skill in the art that other MCT formulations can be used in the biopharmaceutics modeling system and methods disclosed herein, and that AC-SD-03 is merely an example of one such formulation.

In some instances, the MCT formulation (e.g., AC-SD-03) can be characterized with regard to dispersibility and digestion behavior in vitro.

In some instances, the dispersibility studies may include a method of evaluating dispersibility and lipolysis of a medium chain triglyceride (MCT). The evaluation determines physical stability and gastric emptying of a formulation comprising MCT in the stomach, using a simulated gastric medium. The method may comprise dispersing the formulation in gastric medium, incubating the formulation without agitation, for 3.5 to 4.5 hours at 36° C. to 38° C., obtaining samples of the supernatant from the formulation at intervals of one or more of 0 minutes, 5 minutes, 10 minutes, 15 minutes, 30 minutes, 45 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, and 4.5 hours, quantifying the amount of MCT in each sample, analyzing the amount of MCT quantified to determine the time when sedimentation of the MCT in the formulation occurs, wherein the time of sedimentation indicates gastric retention, bioavailability of MCT, and/or extent of digestion.

In some instances, any gastric medium known in the art can be used in the method for determining physical stability of formulation. The gastric medium may comprise a phosphate buffer at a pH of 1.2. The phosphate buffer may comprise 50 mM phosphate buffer. The gastric medium may be prepared from a mixture of sodium chloride (2.0 g), dilute hydrochloric acid (24 ml) and water (approximately 1000 ml). In some aspects, the gastric medium was prepared by dissolving pepsin (1 g), gastric mucin (1.5 g), and NaCl (8.775 g) in 1 L distilled water with pH of 1.3 adjusted using 6 N HCl. The gastric medium may be a commercially available artificial gastric fluid or simulated gastric fluid. The gastric medium may comprise a solution that simulates the composition and pH of gastric juice. Non-limiting example of the components in the gastric medium include sodium chloride, dilute acid, pepsin, mucin, etc.

The method may comprise dispersing a formulation comprising MCT in gastric medium. Dispersing the formulation comprises mixing a powder of a formulation comprising MCT in the phosphate buffer or in the artificial gastric medium. Dispersing the formulation may result in a suspension of formulation comprising MCT in the phosphate buffer or in the artificial gastric medium. Incubation may be performed with further agitation. The dispersion may then be incubated without agitation, for 3 to 5 hours, at 35° C. to 39° C.

Incubation may be performed for about 3 hours, about 3.5 hours, about 4 hours, about 4.5 hours, or about 5 hours. In some instances, the incubation may be performed at about 4 hours.

The dispersion may be incubated at a temperature of about 35° C. to about 39° C. In some aspects, the dispersion is incubated at a temperature of about 35° C., about 36° C., about 37° C., about 38° C., or about 39° C. In some aspects, the dispersion is incubated at a temperature of about 37° C.

The method may further comprise obtaining samples of the supernatant from the formulation, following incubation. The samples may be obtained at several intervals. The sample may be obtained at start of the incubation, at 0 minutes, and at one or more intervals. The one or more intervals may comprise obtaining samples at 0 minutes, 5 minutes, 10 minutes, 15 minutes, 30 minutes, 45 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, and/or 4.5 hours.

The method may further comprises quantifying the amount of MCT in the samples obtained at several intervals. Quantifying MCT in the sample may comprise quantifying using a colorimetric assay. Any known quantitative colorimetric or fluorometric assay kits commercially available can be used for quantifying MCT, in the disclosed method (for e.g., Sigma-Aldrich MAK266-1KT). During the assay, triglycerides may be converted to free fatty acids and glycerol, through chemical hydrolysis, enzymatic hydrolysis, or any combination thereof. In some instances, chemical hydrolysis can be performed by adding an alcoholic solution of alkali metal hydroxide. In some instances, enzymatic hydrolysis can be performed using enzymes non-limiting examples of which include lipase, cholesterol esterase, carboxylesterase, protease, or any combination thereof. Glycerol is then oxidized to generate a product which reacts with a probe to generate color and fluorescence, detected using a spectrophotometer. Glycerol may be determined by enzymatically converting the glycerol to glycerol-1-phosphate with ATP and Mg⁺⁺, enzymatically converting the glycerol-1-phosphate to dihydroxyacetone phosphate with oxidized nicotinamide adenine dinucleotide (NAD), thereby reducing the NAD to reduced form, NADH, and determining the amount of NADH formed. The NADH may be determined by reacting it in the presence of an electron carrier with a colorless tetrazolium dye adapted to form a colored product in such reaction, and photometrically determining the amount of resulting colored tetrazolium product.

The method may further comprise measuring the time when sedimentation of the MCT in the formulation occurs. The time when sedimentation of the MCT in the formulation occurs can be measured qualitatively by visual means.

The amount of MCT quantified and time when sedimentation of the MCT in the formulation occurs, can be utilized to determine gastric retention, bioavailability, and/or extent of digestion of MCT.

In some instances, the dispersibility studies for AC-SD-03 can be carried out in 50 mM phosphate buffer at pH 1.2. The powder (0.5 g) can be dispersed in 8 mL of phosphate buffer and incubated without further agitation for 4 h at 37° C. To assess the stability, 0.1 mL aliquots of the supernatant can be collected at 0, 5, 10, 30 min, 1, 2, 3, and 4 h, respectively. The tricaprilin amount can be measured using the triglyceride quantification colorimetric kit (n=3).

In some instances, the present disclosure provides a method of determining the digestion of a formulation comprising an MCT. The disclosed method may comprise incubating the formulation comprising MCT in gastric medium for about 10 minutes to about 60 minutes at 35° C. to 39° C. with constant stirring, obtaining samples of the formulation in gastric medium at constant intervals over the 30 minutes and adding lipase inhibitor to each of the collected samples at the time of collection, after the 30 minute incubation adjusting the pH of the formulation in gastric medium to 6.80, adding FaSSIF solution to the pH adjusted formulation, initiating lipolysis in the formulation by adding lipase to the formulation, incubating the formulation for 60 minutes while maintaining a pH of 6.8, obtaining samples of the formulation prior to the incubation and then at constant intervals over the 60 minute incubation and adding lipase inhibitor to each of the collected samples at the time of collection, quantifying the amount of digested MCT in each sample optionally using HPLC, and analyzing the quantification information to determine the digestion rate, extent, and/or profile of the formulation comprising MCT.

Any gastric medium known in the art can be used in the method for determining digestion of a formulation. The gastric medium may comprise a phosphate buffer at a pH of 1.2. THe phosphate buffer may comprise 50 mM phosphate buffer. The gastric medium may be prepared from a mixture of sodium chloride (2.0 g), dilute hydrochloric acid (24 ml) and water (approximately 1000 ml). The gastric medium may be prepared by dissolving pepsin (1 g), gastric mucin (1.5 g), and NaCl (8.775 g) in 1 L distilled water with pH of 1.3 adjusted using 6 N HCl. The gastric medium may be a commercially available artificial gastric fluid or simulated gastric fluid. The gastric medium may comprise a solution that simulates the composition and pH of gastric juice. Non-limiting example of the components in the gastric medium include sodium chloride, dilute acid, pepsin, mucin etc.

The formulation may be incubated about 10 minutes to about 60 minutes at 36° C. to 38° C. with constant stirring. The formulation may be incubated for about 10 minutes, about 15 minutes, about 20 minutes, about 25 minutes, about 30 minutes, about 35 minutes, about 40 minutes, about 45 minutes, about 50 minutes, about 55 minutes, or about 60 minutes. The formulation may be incubated about 30 minutes. The formulation may be incubated at a temperature of about 35° C. to about 39° C. The formulation may be incubated at a temperature of about 35° C., about 36° C., about 37° C., about 38° C., or about 39° C. In some instances, the formulation may be incubated at a temperature of about 37° C.

As disclosed herein, the method may further comprise obtaining samples of the formulation in gastric medium at constant intervals over the 30 minutes and adding lipase inhibitor. The samples may be obtained at about 0 minutes, about 5 minutes, about 10 minutes, about 15 minutes, about 20 minutes, about 25 minutes, and/or about 30 minutes. The samples may be obtained at about 0 minutes, about 15 minutes, and about 30 minutes. The lipase inhibitor may comprise Orlistat, 4-BPBA, lipstatin, panclicins, hesperidin, ebelactones, esterastin, valilactone, benzoxazinone, or derivatives thereof. The lipase inhibitor may comprise 4-BPBA. In some instances, about 1 μL to about 5 μL of 0.5 M of lipase inhibitor may be added. In some instances, about 1 μL, about 1.5 μL, about 2 μL, about 2.5 μL, about 3 μL, about 3.5 μL, about 4 μL, about 4.5 μL, or about 5 μL of lipase inhibitor is added. In some instances, 2.5 μL of 0.5 M 4-BPBA in methanol of lipase inhibitor may be added.

The disclosed method may further comprise adjusting the pH of the formulation in gastric medium to about 6.80. For e.g., 5 M NaOH solution can be used to adjust the pH to about 6.80.

According to certain instances of the method, Fasted State Simulated Intestinal Fluid (FaSSIF) solution is used to simulate intestinal conditions. FaSSIF solution can be prepared by mixing 3 mM Sodium taurocholate, 0.75 mM Lecithin, 0.174 g of NaOH (pellets), 1.977 g of NaH2PO4·H2O or 1.719 g of anhydrous NaH2PO4, and 3.093 g of NaCl in 500 mL of purified water. The FaSSIF solution may have a pH of about 6.5 and an osmolality of about 270 mOsmol/kg. Commercially available FaSSIF (for e.g., Biorelevant V2FAS) may be used for the disclosed method. About 1-4 mL of 10× concentrated FaSSIF solution may be added. About 1 ml, about 2 ml, about 3 ml or about 4 ml of 10× concentrated FaSSIF solution may be added. About 2 ml of 10× concentrated FaSSIF solution is added.

In some instances, the method may comprise initiating lipolysis in the formulation by adding lipase to the formulation, and incubating the formulation for 60 minutes while maintaining a pH of 6.8. Examples of lipases suitable for the present invention include, but are not limited to animal lipase (e.g., porcine lipase, pancreatin lipase), bacterial lipase (e.g., Pseudomonas lipase and/or Burkholderia lipase), fungal lipase, plant lipase, recombinant lipase (e.g., produced via recombinant DNA technology by a suitable host cell, selected from any one of bacteria, yeast, fungi, plant, insect or mammalian host cells in culture, or recombinant lipases, which include an amino acid sequence that is homologous or substantially identical to a naturally occurring sequence, lipases encoded by a nucleic acid that is homologous or substantially identical to a naturally occurring lipase-encoding nucleic acid, etc.), synthetic lipase, chemically modified lipase, and mixtures thereof. In some instances, the lipase is a pancreatin lipase suspension. The lipase suspension can be prepared using any known methods in the art. For e.g., lipase can be dispersed in an aqueous solvent (e.g., water, acetone, ethanol). In some instances, about 1-4 ml of lipase may be added to the formulation. In some instances, about 1 ml, about 2 ml, about 3 ml or about 4 ml of the lipase may be added to the formulation. In some instances, about 2 ml of the lipase may be added to the formulation.

During or after initiation of lipolysis, the method may further comprise obtaining samples. The samples may be obtained at 0 minutes, 5 minutes, 10 minutes, 15 minutes, 20 minutes, 25 minutes, 30 minutes, 35 minutes, 40 minutes, 45 minutes, 50 minutes, 55 minutes, and 60 minutes. The samples may be further processed. In some instances, samples are centrifuged and supernatant may be used for further analysis.

The method may further comprise analysis of the free fatty acid released by the digestion. Any know method in the art may be used, including high performance liquid chromatography (HPLC), gas chromatography (GC), or liquid chromatography-mass spectrometry (LC-MS) to quantify octanoic acid digested formulation. The free fatty acid may be quantified using HPLC. Briefly, chromatographic separation of free fatty acid (such as octanoic acid) is performed at 40° C. on a Waters Symmetry C₁₈ column (3.5 μm, 100 Å, 4.6×75 mm) with a Phenomenex Security Guard Cartridge C₁₈. The free fatty acid is then assayed using isocratic elution with a flow rate of 1 mL/min. The mobile phase can comprise a mixture of 0.1% trifluoroacetic acid in water and methanol (40:60). The free fatty acid (e.g., octanoic acid) is detected at about 220 nm. The injection volume that can be used may be about 10 μL, the run time may be about 10 min and the retention time for the free fatty acid (e.g., octanoic acid) may be about 5.9 min. A free fatty acid stock solution (such as octanoic acid stock solution in water:methanol (40:60)) can be used to prepare a standard reference range of 0.01-2.00 mg/mL in the same solvent. Calculation of the free fatty acid concentration from the peak areas can be conducted based on the calibration curve.

In some aspects, the quantification of free fatty acid (e.g., octanoic acid) can be used to understand the digestion rate and/or digestion profile of the disclosed MCT formulations.

In some instances, to evaluate the release of octanoic acid and simulate gastric conditions, the formulation, AC-SD-03, (2.5 g) can be added to 20 mL of 50 mM phosphate buffer at pH 1.2 and vortexed thoroughly. The formulation can be incubated in a 70-mL capacity thermostated glass vessel (37° C.) under constant magnetic stirring for 30 min. Samples (100 μL) can be collected into Eppendorf tubes containing 2.5 μL of lipase inhibitor (0.5 M 4-BPBA in methanol) and 147.7 μL methanol and vortexed for 30 s at 0, 15, and 30 min. The pH of the mixture can be then adjusted to 6.800±0.003 with 5 M NaOH solution. To simulate intestinal conditions, 2 mL of 10× concentrated FaSSIF solutions can be added to the vessel. Lipolysis can be initiated by the addition of pancreatin lipase suspension (2 mL). The pancreatin lipase suspension can be prepared from pancreatin extract using the method described previously [26]. The pH of the sample in the digestion vessel can be maintained at 6.8 during digestion using a 2 M NaOH solution titrated into the digestion medium by a pH-stat control module (Metrohm AG, Herisau, Switzerland). Before digestion (0 min time point) a sample (100 μL) can be collected into an Eppendorf tube containing lipase inhibitor (0.5 M 4-BPBA in methanol) (2.5 μL) and methanol (147.7 μL) and vortexed for 30 seconds, thereafter samples can be collected at 5-min intervals for 60 min. Samples can be then centrifuged at 16142 g for 30 min. The resultant supernatant can be transferred into HPLC vials for analysis of the octanoic acid released by the digestion. The experiment can be conducted in triplicate.

In some instances, the biopharmaceutics modeling system 100 can perform quantification of free fatty acid from the digestion samples. In some instances, the free fatty acid is octanoic acid. The digested formulations can be analyzed on a Shimadzu (Shimadzu, Kyoto, Japan) UPLC system consisting of a CBM-20A system controller, LC-30AD solvent delivery module, SIL-30AC autosampler, and a CTO-20AC column oven coupled to an SPD-M30A diode array. Chromatographic separation of octanoic acid can be performed at 40° C. on a Waters Symmetry C18 column (3.5 μm, 100 A, 4.6×75 mm) with a Phenomenex Security Guard Cartridge C18. Octanoic acid can be assayed using isocratic elution with a flow rate of 1 mL/min. The mobile phase consisted of a mixture of 0.1% trifluoroacetic acid in water and methanol (40:60). Octanoic acid can be detected at 220 nm. The injection volume can be 10 μL, the run time can be 10 min and the retention time for octanoic acid can be 5.9 min. The octanoic acid stock solution in water:methanol (40:60) can be used to prepare a standard reference range of 0.01-2.00 mg/mL in the same solvent. The detection limit and quantification limit for the HPLC method can be 0.002 and 0.006 mg/mL, respectively. Calculation of the octanoic acid concentration from the peak areas can be conducted based on the calibration curve with R2>0.999. It will be appreciated by those of ordinary skill in the art that other free fatty acids can be used in the biopharmaceutics modeling system and methods disclosed herein, and that octanoic acid is merely an example.

In some scenarios, the biopharmaceutics modeling system 100 can perform extraction and analysis of literature data to generate the training data 122 for the PBB model 102. The plasma concentration-time profiles selected from the literature can be extracted using WebPlotDigitizer (v4.3, Ankit Rohatgi, USA). For pharmacokinetic analysis, model design, and data simulations. MonolixSuiteD4 2020R1 (Lixoft, Antony, France), as depicted in FIG. 2.

A clinical pharmacokinetic study can be conducted (e.g., to generate the model inputs 106) with the MCT formulation. For example, a clinical pharmacokinetic study was conducted on the tricaprilin product AC-SD-03, approved by the local Institutional Review Board and carried out in compliance with the Good Clinical Practice guidelines of the International Conference of Harmonization (ICH-GCP). It was an open-label, randomized 2-way crossover study. Twenty healthy male (Caucasian and Chinese) volunteers in an age range of 18-50 years were enrolled. All subjects had undergone a supervised overnight fast of at least 8 h until 1 h before their scheduled morning dose. The breakfast consisting of 2 pieces of toast with butter, 2 strips of bacon, 2 scrambled eggs, 113 g of hash browns, and 240 mL of whole milk was standardized for all subjects. AC-SD-03 (50 g, equivalent to 42,500 μmol tricaprilin) dispersed in 240 ml of water was administered to each subject 30 min after the completion of the standard breakfast. An additional 60 ml of water was used to rinse the dosing cup and administered to the subjects. Water was only restricted 1 h before and 1 h after drug administration, but was allowed ad libitum at all other times. Food and other fluid intake was restricted throughout the confinement period. Upon peroral administration of a single dose of the formulation, the blood tricaprilin, octanoic acid, and ketone body concentrations were quantified. For tricaprilin, blood samples were collected at 0, 1.5, 2.0, 2.5, and 8 h. For octanoic acid and ketones (f3-hydroxybutyrate and acetoacetate), blood samples were collected at 0, 0.5, 1.0, 1.5, 2.0, 2.5, 4.0, and 8 h after dosing, respectively. It will be appreciated by those of ordinary skill in the art that other MCT formulations can be used in the biopharmaceutics modeling system and methods disclosed herein, and that AC-SD-03 is merely an example of one such formulation.

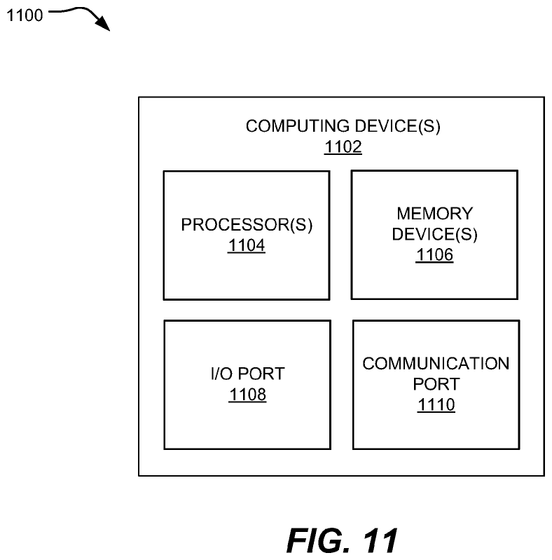

FIG. 11 illustrates an example system 1100 for implementing the biopharmaceutics modeling system 100 with the PBB model 102. The system 1100 can include one or more computer device(s) 1102 which can implement the systems 100-1000 and/or perform the methods discussed herein. In one implementation, the one or more computing device(s) 1102 include a clinic device of the biopharmaceutics modeling system 100, or any other devices discussed throughout this disclosure.

In some instances, the computing device(s) 1102 includes a computer, a personal computer, a desktop computer, a laptop computer, a terminal, a workstation, a cellular or mobile phone, a mobile device, a smart mobile device a tablet, a wearable device (e.g., a smart watch, smart glasses, a smart epidermal device, etc.) a multimedia console, a television, an Internet-of-Things (IoT) device, a smart home device, a medical device, a virtual reality (VR) or augmented reality (AR) device, a vehicle (e.g., a smart bicycle, an automobile computer, etc.), combinations thereof, and/or the like. It will be appreciated that specific implementations of these devices may be of differing possible specific computing architectures not all of which are specifically discussed herein but will be understood by those of ordinary skill in the art.

The computing device 1102 may be a computing system capable of executing a computer program product to execute a computer process. The PBB model 102 can be stored and executed at the computing device(s) 1102 (e.g., as one or more software components, algorithm modules, or so forth). Data and program files may be input to the computing device 1102 which reads the files and executes the programs therein to provide the various components of the PBB model 102 and generate the predictions discussed herein. Some of the elements of the computing device 1102 include one or more hardware processors 1104, one or more memory devices 1106, and/or one or more ports, such as input/output (IO) port(s) 1108 and communication port(s) 1110. Various elements of the computing device 1102 may communicate with one another by way of the communication port(s) 1110 and/or one or more communication buses, point-to-point communication paths, or other communication means.

The processor 1104 may include, for example, a central processing unit (CPU), a microprocessor, a microcontroller, a digital signal processor (DSP), and/or one or more internal levels of cache. There may be one or more processors 1104, such that the processor 1104 comprises a single central-processing unit, or a plurality of processing units capable of executing instructions and performing operations in parallel with each other, commonly referred to as a parallel processing environment.

The computing device 1102 may be a conventional computer, a distributed computer, or any other type of computer, such as one or more external computers made available via a cloud computing architecture. The presently described technology can be optionally implemented in software stored on the data storage device(s) such as the memory device(s) 1106, and/or communicated via one or more of the ports 1108 and 1110, thereby transforming the computing device 1102 in FIG. 11 to a special purpose machine for implementing the operations of the PBB model 102 providing the computer-generated 3D space.

The one or more memory device(s) 1106 may include any non-volatile data storage device capable of storing data generated or employed within the computing device 1102, such as computer executable instructions for performing a computer process, which may include instructions of both application programs and an operating system (OS) that manages the various components of the computing device 1102. The memory device(s) 1106 may include, without limitation, magnetic disk drives, optical disk drives, solid state drives (SSDs), flash drives, and the like. The memory device(s) 1106 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) 1106 may include volatile memory (e.g., dynamic random-access memory (DRAM), static random-access memory (SRAM), etc.) and/or non-volatile memory (e.g., read-only memory (ROM), flash memory, etc.).

Computer program products containing mechanisms to effectuate the systems and methods in accordance with the presently described technology may reside in the memory device(s) 1106 which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computing device 1102 includes one or more ports, such as the I/O port 1108 and the communication port 1110, for communicating with other computing, network, or vehicle devices. It will be appreciated that the I/O port 1108 and the communication port 1110 may be combined or separate and that more or fewer ports may be included in the computing device 1102.

The I/O port 1108 may be connected to an I/O device, or other device, by which information is input to or output from the computing device 1102. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing device 1102 via the I/O port 1108. Similarly, the output devices may convert electrical signals received from the computing device 1102 via the I/O port 1108 into signals that may be sensed as output by a human, such as sound, light, and/or touch. The input device may be an alphanumeric input device, including alphanumeric and other keys for communicating information and/or command selections to the processor 1104 via the I/O port 1108. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

In one implementation, the communication port 1110 can be connected to a network and the computing device 1102 may receive network data useful in executing the methods and systems set out herein as well as transmitting information and network configuration changes determined thereby. Stated differently, the communication port 1110 can connect the computing device 1102 to one or more communication interface devices configured to transmit and/or receive information between the computing device 1102 and other devices by way of one or more wired or wireless communication networks or connections. Examples of such networks or connections include, without limitation, Universal Serial Bus (USB), Ethernet, Wi-Fi, Bluetooth®, Near Field Communication (NFC), and so on. One or more such communication interface devices may be utilized via the communication port 1110 to communicate one or more other machines, either directly over a point-to-point communication path, over a wide area network (WAN) (e.g., the Internet), over a local area network (LAN), over a cellular network (e.g., third generation (3G), fourth generation (4G), Long-Term Evolution (LTE), fifth generation (5G), etc.) or over another communication means. Further, the communication port 1110 may communicate with an antenna or other link for electromagnetic signal transmission and/or reception.

In an example implementation, the PBB model 102 may be embodied by instructions stored on the memory devices 1106 and executed by the processor 1104.

The system 1100 set forth in FIG. 11 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized. In the present disclosure, the methods disclosed may be implemented as sets of instructions or software readable by the computing device 1102.

FIG. 1200 illustrates an example method for predicting pharmacokinetic parameters following administration of a formulation, which can be performed by any of the systems 100-1100 disclosed herein.

In some examples, at operation 1202, the method 1200 can generate training data indicating human metabolism outcomes of the formulation, the human metabolism outcomes including at least one of stomach emptying parameters, a digestion rate, an absorption constant, or a conversion rate, with a training target variable being plasma free fatty acid (such as octanoic acid) concentration or ketone concentration. At operation 1204, the method 1200 can train, by one or more computer devices executing computer-readable instructions, a PBB model with the training data. At operation 1206, the method 1200 can vary, as part of a parameter sensitivity analysis of the PBB model, input parameters of the training data against a simulated mean plasma free fatty acid (e.g., octanoic acid) profile or a simulated mean ketone plasma profile. At operation 1208, the method 1200 can receive, by the one or more computer devices, patient data corresponding to a patient that receives the formulation perorally. At operation 1210, method 1200 can predict, with the PBB model using the patient data, at least one of a target plasma free fatty acid (such as octanoic acid) concentration or a target ketone concentration.

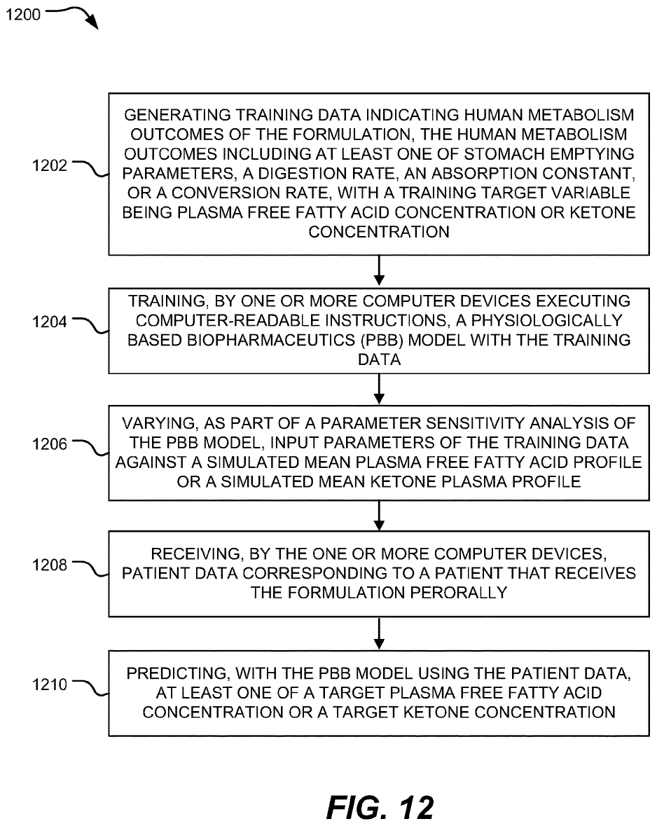

It is to be understood that the specific order or hierarchy of steps in the method 1200 depicted in FIG. 12 or throughout this disclosure are instances of example approaches and can be rearranged while remaining within the disclosed subject matter. For instance, any of the operations depicted in FIG. 12 or throughout this disclosure may be omitted, repeated, performed in parallel, performed in a different order, and/or combined with any other of the operations depicted in FIG. 12 or throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method of predicting pharmacokinetic parameters following administration of a formulation comprising medium-chain triglycerides (MCTs), the method comprising:
generating training data indicating human metabolism outcomes of the formulation;
training, by one or more computer devices executing computer-readable instructions, a physiologically based biopharmaceutics (PBB) model with the training data;
varying, as part of a parameter sensitivity analysis of the PBB model, input parameters of the training data against a simulated mean plasma free fatty acid profile or a simulated mean ketone plasma profile;
receiving, by the one or more computer devices, patient data corresponding to a patient that receives the formulation perorally; and
predicting, with the PBB model using the patient data, at least one of a target plasma free fatty acid concentration or a target ketone concentration.

2. The method of claim 1, wherein the formulation includes a medium-chain triglyceride (MCT) composed of 8-carbon fatty acids with an aqueous solubility of 0.4 mg/L and the target plasma free fatty acid concentration is a target plasma octanoic acid concentration.

3. The method of claim 1, wherein the training data includes a data set based on a gastric emptying model.

4. The method of claim 3, wherein the gastric emptying model includes a sine algorithm corresponding to a rhythmic emptying pattern.

5. The method of claim 3, wherein the gastric emptying model includes a first pathway for solid foods and a second pathway for liquid foods.

6. The method of claim 3, wherein the gastric emptying model includes a stomach amplitude parameter, a complete emptying parameter, and a stomach period parameter.

7. The method of claim 1, wherein predicting, at least one of the target plasma free fatty acid concentration or the target ketone concentration includes determining a digestion rate constant obtained by fitting a release profile of the formulation in a presence of lipases while assuming a first-order kinetic process.

8. The method of claim 1, wherein the training data includes an in vitro parameter being a digestion rate or a bioavailability digestion fraction.

9. The method of claim 1, wherein the training data includes in vivo parameters being at least one of a metabolized fraction, an absorption constant, a conversion rate, an elimination rate, a volume distribution of free fatty acid, or a volume distribution of ketone bodies.

10. The method of claim 1, further comprising performing a dispersibility study for the MCTs to determine a physical stability of the MCTs, predicting at least one of the target plasma free fatty acid concentration or the target ketone concentration is based on the dispersibility study.

11. The method of claim 1, further comprising:
modeling a digestion rate using a release profile of the formulation; and
obtaining a digestion rate constant based on modeling the digestion rate, wherein predicting at least one of the target plasma free fatty acid concentration or the target ketone concentration includes using, with the PBB model, the digestion rate.

12. The method of claim 11, wherein predicting the at least one of the target plasma free fatty acid concentration or the target ketone concentration includes predicting, with the PBB model, a bioavailability of free fatty acid for the patient based on the digestion rate.

13. The method of claim 1, wherein training the PBB model includes instructing the PBB model to consider an enzymatic conversion of free fatty acid to plasma ketones as a non-saturable first-order process.

14. A method of predicting pharmacokinetic parameters following administration of a formulation, the method comprising:
generating training data indicating one or more stomach emptying parameters, a digestion rate, an absorption constant, or a conversion rate with a training target variable being ketone concentration;
training, by one or more computer devices executing computer-readable instructions, a physiologically based biopharmaceutics (PBB) model with the training data;
receiving, by the one or more computer devices, patient data corresponding to a patient that receives the formulation; and
predicting, with the PBB model using the patient data, a target ketone concentration.

15. The method of claim 14, wherein predicting the target ketone concentration includes predicting, with the PBB model and using the patient data, a plasma free fatty acid concentration of the patient.

16. The method of claim 14, further comprising performing a validation for the PBB model using clinical trial data which quantifies free fatty acid and ketone bodies from blood plasma of volunteers.

17. A method of predicting pharmacokinetic parameters following administration of a formulation comprising medium chain triglycerides (MCTs), the method comprising:
generating training data indicating stomach emptying parameters, a digestion rate, an absorption constant, or a conversion rate with a training target variable being plasma free fatty acid concentration;
training, by one or more computer devices executing computer-readable instructions, a physiologically based biopharmaceutics (PBB) model with the training data;
receiving, by the one or more computer devices, patient data corresponding to a patient that receives the formulation; and
predicting, with the PBB model using the patient data, a target plasma free fatty acid concentration.

18. The method of claim 17, further comprising predicting a ketone concentration with the PBB model using the patient data, the target plasma free fatty acid concentration, and a conversion rate estimate.

19. The method of claim 17, wherein training the PBB model includes providing a gastric absorption emptying model having a sine algorithm with an amplitude variable, a period variable, and a complete emptying time variable.

20. The method of claim 17, wherein the training data includes a first cluster including physiologically based parameters, a second cluster including population parameters, and a third cluster including in vitro parameters.
